(19) United States
(12) Patent Application Publication
(10) Pub. No.: US 2026/0096790 A1
(43) Pub. Date: Apr. 9, 2026

(54) DISTRIBUTED MEDICAL IMAGE ACQUISITION

(71) Applicant: Siemens Medical Solutions USA, Inc., Malvern, PA (US)

(72) Inventors: Alexander Hans Vija, Evanston, IL (US); Andrew Scheffel, Grayslake, IL (US)

(21) Appl. No.: 19/115,508

(22) PCT Filed: Jul. 20, 2023
(86) PCT No.: PCT/US2023/070565

§ 371 Date: Mar. 26, 2025

Related U.S. Application Data
(60) Provisional application No. 63/478,514, filed on Jan. 5, 2023.

(51) Int. Cl. A61B 6/00; A61B 6/03; A61B 6/42

(52) U.S. Cl. CPC A61B 6/4405; A61B 6/037; A61B 6/4241; A61B 6/482; A61B 6/5235; A61B 6/547; A61B 6/56

(57) ABSTRACT

A framework for distributed medical image acquisition. An optimal configuration of one or more mobile imaging systems to address a clinical task is determined. The one or more mobile imaging systems may be dispatched in accordance with the optimal configuration to perform medical image acquisition of a patient to generate medical image data. Image reconstruction may then be performed based on the medical image data.

### CROSS-REFERENCE TO RELATED APPLICATION

This application claims the benefit of U.S. Provisional Application No. 63/478,514, filed Jan. 5, 2023, entitled “Cooperative SPECT: Operation and Optimization of Multi Modal Workflow with Mobile SPECT Units,” which is herein incorporated by reference.

### TECHNICAL FIELD

The present disclosure generally relates to medical imaging, and more particularly to mobile imaging systems for distributed medical image acquisition.

### BACKGROUND

The field of medical imaging has seen significant advances since the time X-Rays were first used to determine anatomical abnormalities. Medical imaging hardware has progressed in the form of newer machines such as Medical Resonance Imaging (MRI) scanners, Computed Axial Tomography (CAT) scanners, etc. Digital medical images are constructed using raw image data obtained from such scanners.

Typically, only one imaging system is assigned to one imaging suite for imaging one patient at a time. Throughput cannot be increased beyond the imaging performance of the specific imaging system. Furthermore, technology updates to address imaging performance issues (i.e., a “forklift” upgrade) require an exchange of imaging systems, often incurring construction cost and delay. This approach requires a business model that is not flexible and is sub-optimal from an operational standpoint, since most imaging systems are idle, with utilization of less than 50% per day.

Injection of the patient occurs in the injection room, and the patient needs to go to the imaging suite thereafter. The patient may be ambulated or transported to the imaging suite, imaged and either released or transferred back to the station. This imaging process is performed for each imaging modality. It is a costly logistical problem as patients must be scheduled and transported from room to room. In addition, the design of the imaging system is not optimal for all imaging tasks. It is often designed as either as a general purpose or a dedicated system, and not designed to cooperate with other imaging systems. When the imaging system needs to be shut down for maintenance service, such service typically occurs in “patient”space and therefore interrupts the workflow.

Currently, there is no solution other than to bring the patient to the respective imaging systems, even if that means to another hospital. Small footprint imaging system designs can address the footprint issues, but these imaging systems with smaller footprints typically include dedicated organ cameras or specialty cameras (e.g., thyroid scintigraphy), and are not designed for tomography. Other designs with ultra-small footprint and portability are designed as gamma probe with very small imaging fields to assist in, for example, surgery of sentinel node biopsy.

### SUMMARY

Described herein is a framework for distributed medical image acquisition. An optimal configuration of one or more mobile imaging systems to address a clinical task is determined. The one or more mobile imaging systems may be dispatched in accordance with the optimal configuration to perform medical image acquisition of a patient to generate medical image data. Image reconstruction may then be performed based on the medical image data.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings.

FIG. 1 is a block diagram illustrating an exemplary mobile imaging system;

FIG. 2 shows an exemplary method of distributed image acquisition;

FIG. 3 shows exemplary clinical use cases;

FIG. 4 depicts an exemplary situation where the mobile imaging system is moved to an arena where typical single-photon emission computed tomography (SPECT) imaging is not possible; and

FIG. 5 depicts an exemplary use case where mobile imaging systems assembled themselves for optimal axial coverage.

### DETAILED DESCRIPTION

In the following description, numerous specific details are set forth such as examples of specific components, devices, methods, etc., in order to provide a thorough understanding of implementations of the present framework. It will be apparent, however, to one skilled in the art that these specific details need not be employed to practice implementations of the present framework. In other instances, well-known materials or methods have not been described in detail in order to avoid unnecessarily obscuring implementations of the present framework. While the present framework is susceptible to various modifications and alternative forms, specific embodiments thereof are shown by way of example in the drawings and will herein be described in detail. It should be understood, however, that there is no intent to limit the invention to the particular forms disclosed, but on the contrary, the intention is to cover all modifications, equivalents, and alternatives falling within the spirit and scope of the invention. Furthermore, for ease of understanding, certain method steps are delineated as separate steps; however, these separately delineated steps should not be construed as necessarily order dependent in their performance. Independent of the grammatical term usage, individuals with male, female or other gender identities are included within the term.

Unless stated otherwise as apparent from the following discussion, it will be appreciated that terms such as “segmenting,” “generating,” “registering,” “determining,” “aligning,” “positioning,” “processing,” “computing,” “selecting,” “estimating,” “detecting,” “tracking” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods can be compiled for execution on a variety of hardware platforms and for interface to a variety of operating systems. In addition, implementations of the present framework are not described with reference to any particular programming language. It will be appreciated that a variety of programming languages may be used.

A framework for distributed medical image acquisition is presented herein. In accordance with one aspect, mobile imaging systems with specific imaging parameters are provided for facilitating distributed image acquisition. In some implementations, one or more of the mobile imaging systems autonomously move to the patient's bedside for performing medical image acquisition of the patient. Additionally, the mobile imaging systems may cooperate with each other and assemble to optimally address the clinical task. “Cooperation” as used herein generally refers to the mobile imaging systems working together and sharing data (e.g., sensor data, medical image data) to accomplish the common goal of optimizing the completion of the clinical task. The mobile imaging systems may assemble using a common communication protocol. One of the mobile imaging systems may be designated as the reference system to align spatial and temporal markers, as well as to acquire and reconstruct image data as one image volume.

The framework advantageously provides distributed image acquisition that allows for extra-and/or intra-modal information to optimally assess the patient. This framework brings clinical workflow and operational changes to clinical workflow to optimize utilization and use of medical imaging devices. Distributed emission acquisition, by simultaneous assessment of gamma ray spectrum from a patient with dedicated specialized units, enables optimal assessment and utilization. Additionally, when one mobile imaging system is down for maintenance, other replacement systems may be dispatched to provide the medical imaging service. The maintenance of the mobile imaging system may occur outside a “patient” space (e.g., within a storage area), thereby minimizing disruption to the workflow. These and other exemplary advantages and features will be described in more details in the following description.

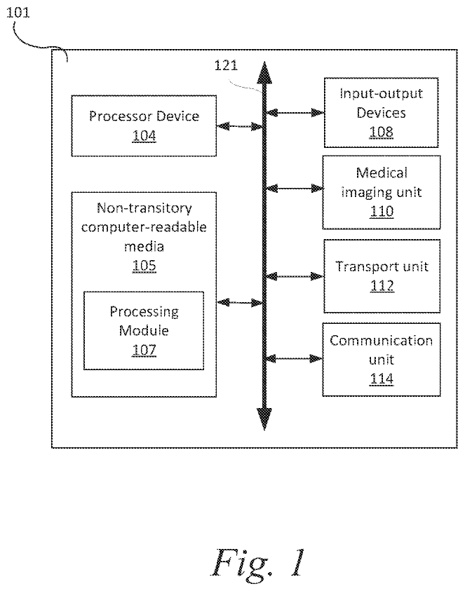

FIG. 1 is a block diagram illustrating an exemplary mobile imaging system 101 for implementing the framework as described herein. In some implementations, mobile imaging system 101 operates as a standalone device. In other implementations, mobile imaging system 101 may be connected via communication module 114 to other machines, such as other mobile imaging systems 101. In a networked deployment, mobile imaging system 101 may operate as a peer machine in a peer-to-peer (or distributed) network environment. Any number of mobile imaging systems 101 may be provided (e.g., two, three or more). Each of the mobile imaging systems 101 may move and/or operate independently of the other mobile imaging systems 101.

Mobile imaging system 101 may include a processor device or central processing unit (CPU) 104 coupled to one or more non-transitory computer-readable media 105 (e.g., computer storage or memory device), input-output devices 108 (e.g., monitor, mouse, touchpad or keyboard), medical imaging unit 110, transport unit 112 and communication unit 114 via an input-output interface 121. Mobile imaging system 101 may further include support circuits such as a cache, a power supply or battery, clock circuits and a communications bus (not shown). Mobile imaging system 101 may be charged at, for example, a docking station in a storage location and/or charging docks distributed throughout the hospital. Corded power may also be provided for operation during scan. Various other peripheral devices, such as additional data storage devices and printing devices, may also be connected to the mobile imaging system 101.

The present technology may be implemented in various forms of hardware, software, firmware, special purpose processors, or a combination thereof, either as part of the microinstruction code or as part of an application program or software product, or a combination thereof, which is executed via the operating system. In some implementations, the techniques described herein are implemented as computer-readable program code tangibly embodied in one or more non-transitory computer-readable media 105. In particular, the present techniques may be implemented by a processing module 107. Non-transitory computer-readable media 105 may include random access memory (RAM), read-only memory (ROM), magnetic floppy disk, flash memory, and other types of memories, or a combination thereof. The computer-readable program code is executed by processor device 104 to process data acquired by, for example, medical imaging unit 110. The computer-readable program code is not intended to be limited to any particular programming language and implementation thereof. It will be appreciated that a variety of programming languages and coding thereof may be used to implement the teachings of the disclosure contained herein. The same or different computer-readable media 105 may be used for storing a database, including, but not limited to, image datasets, a knowledge base, individual subject data, medical records, diagnostic reports (or documents) for subjects, or a combination thereof.

Medical imaging unit 110 acquires medical image data. Medical imaging unit 110 may be a tomography scanner (e.g., nuclear medicine scanner) for acquiring, collecting and/or storing such medical image data. Medical imaging unit 110 may be a single-photon emission computed tomography (SPECT), positron emission tomography (PET), computed tomography (CT) or other tomography (e.g., ultrasound or surgical) imaging system. Diagnostic, theragnostic, dosimetry or surgical support imaging may be provided.

In some implementations, medical imaging unit 110 is a SPECT imaging system that includes a detector connected to, for example, an arm via articulated coupling. The detector may be a gamma ray detector that generates a tomographic image from a fixed position. The detector may include a flat panel or a curved panel. Current examples of such systems include a parallel hole collimator, a rotating acceptance angle collimator in which the aperture passageways in the collimator are arranged so that the angle of view of each row varies with respect to a radiation source, and a rotating slit/slat collimator used in combination with a gamma camera having a scintillation detector formed of a stack of scintillation bar detectors. Other types of imaging systems are also useful. For example, attenuation pattern encoded or non-attenuation pattern encoded, multiplexed or non-multiplexed, time encoded or non-time encoded imaging system are also useful.

When the mobile imaging system 101 is further away from the patient, medical image data with a larger field-of-view of the patient and lower resolution may be acquired. As the mobile imaging system 101 approaches the patient, medical imaging unit 110 may acquire medical image data of the patient with increasing resolution but decreasing field-of-view. Image processing and/or reconstruction may be performed based on the acquired medical image data as the mobile imaging system 101 approaches the patient, allowing the mobile imaging system 101 to display tomographic images that “zoom” into the volume of interest.

Transport unit 112 serves to autonomously or semi-autonomously position the mobile imaging system 101 near the patient for acquiring medical image data. In some implementations, the transport unit 112 includes at least one mobile structure (e.g., wheels, cylinders, rollers, legs) for freely moving the mobile imaging system 101, a drive module for driving the at least one mobile structure and a sensor module. The drive module may include a motor (e.g., electric motor) that may be driven by a human operator or self-driven in response to a signal initiated by processing module 107. The sensor module includes one or more sensors for determining position and/or orientation of the mobile imaging system 101, detecting obstacles to avoid collisions, and/or detecting the position of the patient. The one or more sensors may include, for example, a camera, range sensor, ultrasound sensor, infrared sensor, global positioning sensor, or a combination thereof. See, for example, United States Patent Publication No. US 20210219927A1 and United States Patent Publication No. US 20220015726A1, which are herein incorporated by reference.

Communication unit 114 enables the mobile imaging system 101 to communicate with other mobile imaging systems and/or other systems. Mobile imaging system 101 may communicate with other mobile imaging systems and/or other systems to, for example, cooperate and/or arrange themselves for optimal coverage. Communication unit 114 may include wireless signal transceiver that communicate signals using a common communication protocol, such as Global System for Mobile Communications (GSM), WIFI, Bluetooth, Zigbee, LoRa, and TCP/IP. Other types of communication protocols are also useful. The wireless communications are used to assemble the mobile imaging systems 101 in an optimal configuration to address a clinical task. One of the mobile imaging systems 101 may be designated as a reference system to provide a reference position and/or clock to which the other mobile imaging systems 101 align spatially and/or temporally.

It is to be further understood that, because some of the constituent system components and method steps depicted in the accompanying figures can be implemented in software, the actual connections between the systems components (or the process steps) may differ depending upon the manner in which the present framework is programmed. Given the teachings provided herein, one of ordinary skill in the related art will be able to contemplate these and similar implementations or configurations of the present framework.

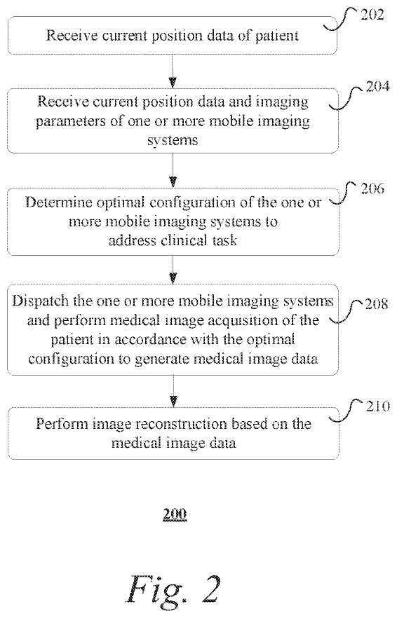

FIG. 2 shows an exemplary method 200 of distributed image acquisition. It should be understood that the steps of the method 200 may be performed in the order shown or a different order. Additional, different, or fewer steps may also be provided. Further, the method 200 may be implemented with at least one mobile imaging system 101 of FIG. 1, a different system, or a combination thereof. For example, the processing module 107 may be located in a mobile imaging system 101 or another computer system that serves as the control system.

At 202, processing module 107 receives current position data of the patient. The current position data of the patient represents the current location of the patient. The current position data of the patient may be acquired by the one or more sensors in the sensor module of the transport unit 112 in one or more mobile imaging systems 101. The current position data of the patient may be provided relative to a floor map of the medical facility. The floor map may be predefined or adaptively learnt based on the sensor data from the sensor module.

At 204, processing module 107 receives current position data and imaging parameters of one or more mobile imaging systems 101. The current position data of one or more mobile imaging systems 101 represents the current location(s) of the one or more mobile imaging systems 101. The current position data of the one or more mobile imaging systems 101 may be acquired by the sensor module and transmitted via the communication unit 114 of each of the one or more mobile imaging systems 101. The current position data of the of one or more mobile imaging systems 101 may be provided relative to the floor map of the medical facility.

Imaging parameters describe the properties of the medical imaging unit 110. Imaging parameters of the one or more mobile imaging systems 101 may be retrieved from non-transitory computer-readable media 105. Alternatively, imaging parameters may be transmitted via the communication unit 114 of each of the one or more mobile imaging systems 101 to the processing module 107. Imaging parameters include, but are not limited to, field-of-view (FOV) (e.g., axial FOV), energy resolution, energy range, spatial resolution, detection efficiency, collimator characteristics, or a combination thereof.

At 206, processing module 107 determines an optimal configuration of the one or more mobile imaging systems 101 to address a clinical task. The optimal configuration may be determined to, for example, best capture emission data emitted by a radioisotope located within a specific portion of the patient. The optimal configuration may be determined based on the current position data of the patient, the current position data of the one or more mobile imaging systems 101, imaging parameters of the one or more mobile imaging systems 101, or a combination thereof. Other extra-modal or intra-modal information may also be used.

The optimal configuration may specify a number of mobile imaging systems 101 to be dispatched. The optimal configuration may also specify positions, imaging parameter values and/or scan times of one or more mobile imaging systems 101 to be assembled for optimal completion of the clinical task. The current position data of the patient may be used to guide the one or more mobile imaging systems 101 to move to the patient. Once the one or more mobile imaging systems 101 are moved next to the patient, medical image acquisition of the patient may be optimized. Patient motion during the image acquisition may also be compensated for. The optimal configuration advantageously enables the one or more mobile imaging systems 101 to assemble when needed at the location where needed, and operate as a single unit or jointly in a fleet to allow for more efficient body coverage when needed. The optimal configuration may be generated by, for example, machine learning techniques (e.g., deep convolutional networks).

The assembly of multiple mobile imaging systems 101, each with different or similar imaging parameters, facilitates the creation of a loose configuration tailored to the clinical task. For example, if only axial field-of-view (FOV_y) expansion is needed for the clinical task, processing module 107 may determine an optimal configuration of k additional mobile imaging systems 101 (wherein k is a positive integer), such that the sum of axial fields-of-view of the mobile imaging systems 101 (i.e., (k+1)*FOV_y) is equal or greater than the desired axial FOV for the patient. The multiple mobile imaging systems 101 may be positioned around the patient such that there is minimal overlap between the axial fields-of-view.

As another example, if a first group of mobile imaging systems 101 performs imaging at, e.g., less than 400 keV, and a second group of mobile imaging systems 101 performs imaging at, e.g., greater than 511 keV, then processing module 107 may determine an optimal configuration in which one or more units are selected from each of the first and second groups. The optimal configuration may specify the scan times and/or locations of the selected mobile imaging systems 101 on the floor map, such that they acquire medical image data simultaneously or sequentially, close to the patient or at a specified distance from the patient to image the whole body of the patient. The imaging panels may optimally “hug” the body contour of the patient to achieve the best possible resolution, yet azimuthally encompass as much as possible to achieve high sensitivity in the respective FOV.

As yet another example, one mobile imaging system 101 may cover a very wide energy range, such as 30 keV to 3000 MeV (e.g., greater than 511 keV) using Compton imaging. The utilization of this mobile imaging system 101 is such that only one unit suffices for the site, whereas multiple mobile imaging systems 101 covering energy ranges within 200-400 keV, and even more mobile imaging systems 101 covering energies <3000 keV may also be available at the site, given the specific site's needs and operation. For example, four mobile imaging systems 101 may cover the 30-400 keV, while two mobile imaging systems 101 cover 511 keV and one mobile imaging system 101 covers 511-3000 keV energy range.

As yet another example, dynamic four-dimensional (4D) image formation may be performed with the mobile imaging systems 101 such that image formation is performed spatial-temporal consistently. The medical imaging unit 110 of such mobile imaging systems 101 may use, for example, a non-multiplexed system, multi-channel collimation system, parallel hole collimator, multiplexed pinhole array, coded aperture or time-encoded aperture, or a combination thereof,

At 208, processing module 107 dispatches the one or more mobile imaging systems 101 to perform medical image acquisition of the patient in accordance with the optimal configuration to generate medical image data. Processing module 107 may initiate the transport unit 112 to autonomously (or semi-autonomously) move the one or more the one or more mobile imaging systems 101 to the respective locations of need indicated by the optimal configuration. Processing module 107 may then initiate the medical imaging unit 110 to perform medical image acquisition.

The transport unit 112 may include a drive module and a sensor module. The drive module may include a motor (e.g., electric motor) that can either be driven by a human operator or autonomously to locations indicated by the optimal configuration. In some implementations, processing module 107 may initiate the drive module to panic move the mobile imaging system 101, so as to rapidly clear the space while avoiding obstacles in response to sensor data from the sensor module. In other implementations, processing module 107 may initiate service mode (e.g., maintenance, calibration) while the mobile imaging system 101 is in storage (e.g., garage space) and/or charging at a power source (e.g., charging dock). Service may occur without interfering with patient operation.

At 210, processing module 107 performs image reconstruction based on the medical image data. Processing module 107 reconstructs a tomographic image of an internal region of interest from the medical image data acquired by one or more mobile image units 101. CT, PET, SPECT, or other types of reconstruction is used. In an iterative optimization, the locations of emissions or the attenuation at the locations is determined from the detected signals. For PET or SPECT, tomographic reconstruction is used to reconstruct the locations of the radioisotope. For CT, tomographic reconstruction is used to reconstruct the attenuation at locations throughout the region of interest.

In some implementations, medical image data from multiple mobile imaging systems 101 is combined to reconstruct a larger volume of interest than is depicted in each of the respective images. The medical image data may be combined by, for example, aligning (or registering) pixels or voxels of one medical image with corresponding pixels or voxels in another medical image that represent the same volume of interest.

Processing module 107 renders the reconstructed tomographic image for display on, for example, a display screen. Alternatively, the image is printed or projected. The image may be combined with a view or other image in an augmented reality display, a virtual display, or a mixed display. Previous images (e.g., CT or MR images) from previous scans may be integrated with the reconstructed tomographic image using registration algorithms.

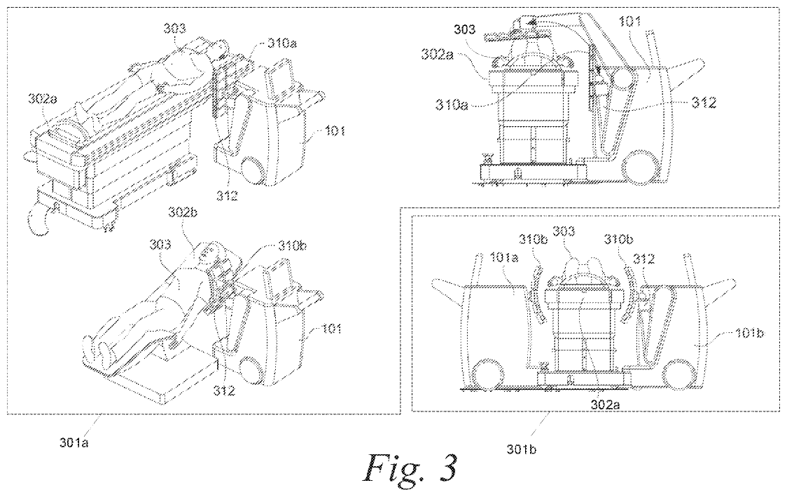

FIG. 3 shows exemplary clinical use cases, wherein the mobile imaging system (101, 101a-b) can handle various types of beds 302a-b in single or multi-unit configurations. More particularly, in the single configuration 301a, bed 302a supports imaging subject 303 in a flat configuration, whereas bed 302b supports imaging subject 303 an inclined configuration. Mobile imaging system 101 may be moved by its transport unit 112 to the desired position with respect to the imaging subject 303. The desired position may be a position according to the optimal configuration determined by processing module 107. The detector 310a-b may be moved to the desired height and/or angular position by the articulating arm 312 to facilitate selective positioning with respect to the imaging subject 303. Detector 310a-b may include multiple cameras that can change from a flat panel configuration (detector 310a) to a curved panel configuration (detector 310b) to optimize image acquisition. In the multi-unit configuration 301b, two mobile imaging systems 101a-b are dispatched to both sides of the bed 302a in a tandem operation.

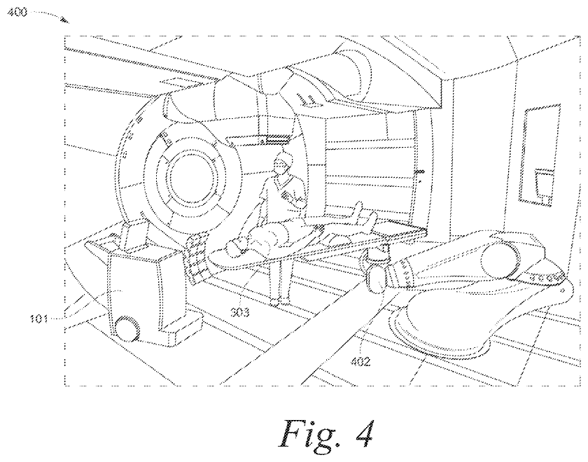

FIG. 4 depicts an exemplary situation 400 where the mobile imaging system 101 is moved to an arena where typical SPECT imaging is not possible. Typical SPECT imaging requires the permanent installation of a large immobile gantry-based system in a large dedicated space. In this case, the arena does not have enough space to allow for such large gantry-based system or even integration with a common patient handling system (PHS) 402. In this case, the PHS 402 is movable on all axis. The mobile imaging system 101 may be dispatched to provide SPECT image acquisition service for any subject of interest 303 without requiring such large space or permanent installation.

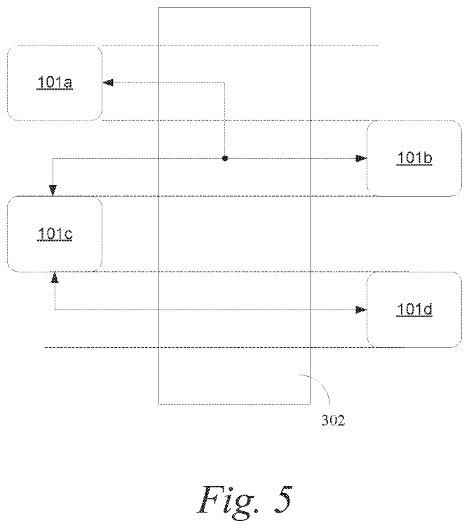

FIG. 5 depicts an exemplary use case wherein mobile imaging systems 101a-d assembled themselves for optimal axial coverage of the subject of interest on the bed 302. The mobile imaging systems 101a-d may exchange information via, for example, Bluetooth or other wireless communication protocols. In this optimal configuration, two mobile imaging systems 101a and 101c are dispatched to one side of the bed 302 and two mobile imaging systems 101b and 101d are dispatched on the other side of the bed 302, such that there is minimal overlap between their axial coverages and the desired axial coverage is achieved. It should be appreciated that other optimal configurations are also possible, such as two mobile imaging systems covering the same axial extent, so as to speed up the acquisition of that field-of-view (FOV).

In addition, the mobile imaging systems 101a-d may have different complementary imaging parameters (e.g., different image formation characteristics), allowing for an optimal assembly of units to address the needed task. For example, one mobile imaging system may perform imaging at high energy (e.g., greater than 511 keV) using a Compton imaging at a distance from the subject of interest, while other mobile imaging systems are close to the patient, with specific image quality characteristics optimally addressing the clinical need.

While the present framework has been described in detail with reference to exemplary embodiments, those skilled in the art will appreciate that various modifications and substitutions can be made thereto without departing from the spirit and scope of the invention as set forth in the appended claims. For example, elements and/or features of different exemplary embodiments may be combined with each other and/or substituted for each other within the scope of this disclosure and appended claims.

What is claimed is:

1. A method of image acquisition, comprising:
receiving first current position data of a patient;
receiving second current position data and imaging parameters of one or more mobile imaging systems;
determining an optimal configuration of the one or more mobile imaging systems to address a clinical task based on the first current position data, the second current position data and the imaging parameters;
dispatching the one or more mobile imaging systems in accordance with the optimal configuration to perform medical image acquisition of the patient to generate medical image data; and
performing image reconstruction based on the medical image data.

2. The method of claim 1 further comprises acquiring the first and second current position data using one or more sensors in the one or more mobile imaging systems.

3. The method of claim 1 further comprises wirelessly transmitting the first and second current position data and the imaging parameters via a communication unit in the one or more mobile imaging systems.

4. The method of claim 1 wherein the imaging parameters comprise a field-of-view (FOV), energy resolution, energy range, spatial resolution, detection efficiency, collimator characteristics, or a combination thereof.

5. The method of claim 1 wherein determining the optimal configuration of the one or more mobile imaging systems comprises determining at least a number, position, imaging parameter value, scan time of the one or more mobile imaging systems, or a combination thereof.

6. The method of claim 1 wherein determining the optimal configuration of the one or more mobile imaging systems comprises determining a configuration of multiple mobile imaging systems, wherein a sum of axial fields-of-view of the multiple mobile imaging systems equals or exceeds a desired axial field-of-view.

7. The method of claim 1 wherein determining the optimal configuration of the one or more mobile imaging systems comprises determining a configuration of multiple mobile imaging systems that perform imaging at different energies.

8. The method of claim 7 wherein at least one of the multiple mobile imaging systems performs imaging at less than 400 ke V and at least another one of the multiple mobile imaging systems performs imaging at greater than 511 keV.

9. The method of claim 1 wherein determining the optimal configuration of the one or more mobile imaging systems comprises determining a configuration of multiple mobile imaging systems positioned around a body contour of the patient.

10. The method of claim 1 wherein dispatching the one or more mobile imaging systems in accordance with the optimal configuration comprises initiating a transport unit of the one or more mobile imaging systems to autonomously move the one or more mobile imaging systems.

11. The method of claim 1 wherein performing the image reconstruction comprises combining tomographic images from multiple mobile imaging systems.

12. A mobile imaging system, comprising:
a medical imaging unit;
a transport unit;
a non-transitory memory device for storing computer readable program code; and
a processor device in communication with the non-transitory memory device, the medical imaging unit and the transport unit, the processor device being operative with the computer readable program code to perform steps including:
determining an optimal configuration to address a clinical task,
initiating the transport unit to move in accordance with the optimal configuration, and
initiating the medical imaging unit to perform medical image acquisition of a patient to generate medical image data.

13. The mobile imaging system of claim 12 wherein the medical imaging unit comprises a single-photon emission computed tomography (SPECT) imaging system.

14. The mobile imaging system of claim 12 wherein the transport unit comprises:
at least one mobile structure;
a drive module for driving the at least one mobile structure; and
a sensor module for determining position data.

15. The mobile imaging system of claim 12 further comprising a communication unit in communication with the processor device that enables communications with other mobile imaging systems.

16. The mobile imaging system of claim 15 wherein the processor device is operative with the computer readable program code to assemble, via the communication unit, the other mobile imaging systems in accordance with the optimal configuration.

17. The mobile imaging system of claim 16 wherein the processor device is operative with the computer readable program code to determine the optimal configuration based on imaging parameters of the mobile imaging system and the other mobile imaging systems.

18. The mobile imaging system of claim 17 wherein the imaging parameters comprise a field-of-view (FOV), energy resolution, energy range, spatial resolution, detection efficiency, collimator characteristics, or a combination thereof.

19. The mobile imaging system of claim 12 wherein the processor device is operative with the computer readable program code to determine the optimal configuration by determining at least a number of other mobile imaging systems to be dispatched.

20. One or more non-transitory computer-readable media embodying instructions executable by a machine to perform operations comprising:
determining an optimal configuration of one or more mobile imaging systems to address a clinical task;
dispatching the one or more mobile imaging systems in accordance with the optimal configuration to perform medical image acquisition of a patient to generate medical image data; and
performing image reconstruction based on the medical image data.
